(19) United States
(12) Patent Application Publication
(10) Pub. No.: US 2012/0027166 A1
(43) Pub. Date: Feb. 2, 2012

(54) RADIATION IMAGE CAPTURING APPARATUS

(75) Inventors: Sadato AKAHORI, Odawara-shi (JP); Yasunori OHTA, Yokohama-shi (JP); Kazuharu UETA, Tokyo (JP); Atsushi FUKUDA, Tokyo (JP)

(73) Assignee: FUJIFILM CORPORATION, Tokyo (JP)

(21) Appl. No.: 13/252,509

(22) Filed: Oct. 4, 2011

Related U.S. Application Data
(63) Continuation of application No. 12/238,878, filed on Sep. 26, 2008, now abandoned.

(30) Foreign Application Priority Data
Sep. 28, 2007 (JP) 2007-255131

(51) Int. Cl. A61B 6/03

(52) U.S. Cl. 378/19

(57) ABSTRACT

A radiation image capturing apparatus includes an overlapping area specifying unit which acquires a plurality of radiation images of the plurality of image capturing ranges of an elongate test subject in advance, specifies overlapping areas in the radiation images, and registers information of the specified overlapping areas in an information table, an image joining unit which acquires a plurality of radiation images of the plurality of image capturing ranges of the elongate subject, and joins the acquired plurality of radiation images based on the information of the overlapping areas registered in the information table to generate a single joined radiation image, and an image generating unit for generating a tomographic image on an arbitrary image plane based on the joined radiation image.

### BACKGROUND OF THE INVENTION

1. Field of the Invention

The present invention relates to a radiation image capturing apparatus including a radiation source and a radiation conversion panel for detecting a radiation emitted from the radiation source and passing through a subject, and converting the detected radiation into radiation image information, and more particularly to a radiation image capturing apparatus for capturing an image of an elongate subject based on tomosynthesis.

2. Description of the Related Art

Radiation image capturing apparatus for capturing images of elongate subjects have been proposed in Japanese Laid-Open Patent Publication Nos. 2004-202069, 2005-066343, and 2006-141904.

Japanese Laid-Open Patent Publication No. 2004-202069 discloses an image reading apparatus for capturing an image of the outer side of a row of teeth as a single image. The image reading apparatus captures the images of a plurality of segments of the outer side of the row of teeth and then accurately combines the segmental images into an overall image of the outer side of the row of teeth. Specifically, the image reading apparatus comprises a digital camera for capturing the segmental images of the outer side of the row teeth segment by segment, a distance sensor for measuring the distance from the digital camera to the outer side of the row of teeth as an image capturing distance, a memory for storing the segmental images and the image capturing distance which is measured when the segmental images are captured, an image magnification converting means for converting the image capturing magnifications of all the segmental images into a life-size magnification based on the image capturing distance, and an image combining means for combining the segmental images into a single combined image.

Japanese Laid-Open Patent Publication No. 2005-066343 discloses a system for acquiring a radiographic tomosynthesis image using asymmetrical geometry, the system having an optimum total sweep angle for maximizing the image quality. The system includes an X-ray detector and an X-ray source capable of emitting X-rays directed at the X-ray detector. For acquiring a radiographic tomosynthesis image, the system utilizes asymmetric image acquisition geometry where θ1≠θ0 where θ1 represents a sweep angle on one side of a center line of the X-ray detector and θ0 a sweep angle on the opposite side of the center line of the X-ray detector.

### SUMMARY OF THE INVENTION

Japanese Laid-Open Patent Publication No. 2006-141904 discloses an X-ray image capturing apparatus which operates as follows: A marker is disposed in an overlapping area on a subject, and the position of the marker is determined from a captured image of the marker. A distance to be traveled up to an image capturing range divided based on the position of the marker is determined by a distance calculating means. A position control means moves an X-ray detecting means to a position corresponding to the distance to be traveled, and automatically holds the X-ray detecting means in the image capturing range. The X-ray image capturing apparatus makes it possible to eliminate a complicated and time-consuming process of positioning a plane detector in capturing an image of an elongate subject.

According to the technology disclosed in Japanese Laid-Open Patent Publication No. 2004-202069, the segmental images captured by the digital camera are joined together into a single image of the outer side of the row of teeth. However, since the two-dimensional images are simply joined together in a planar array, the combined image is far from representing the actual three-dimensional outer side of the row of teeth.

The technology disclosed in Japanese Laid-Open Patent Publication No. 2005-066343 is effective in acquiring a three-dimensional radiographic image in a relatively small range such as a chest or the like. However, the disclosed system is unable to acquire a three-dimensional radiographic image of an elongate area such as the backbone or a leg bone of a human being.

Since the X-ray image capturing apparatus disclosed in Japanese Laid-Open Patent Publication No. 2006-141904 captures a two-dimensional image of an area, the apparatus is disadvantageous in that it needs to recapture another two-dimensional image of the area if the user wants to confirm the area from a different perspective.

It is an object of the present invention to provide a radiation image capturing apparatus which is capable of acquiring a two-dimensional projection image (tomographic image) of an elongate subject on an arbitrary image plane based on a three-dimensional image that is acquired by way of tomosynthesis, for thereby effectively shortening medical procedures that are performed using the radiation image capturing apparatus.

A radiation image capturing apparatus according to the present invention includes a tomosynthesis image capturing device comprising: a radiation source, a radiation conversion panel for detecting a radiation emitted from the radiation source and passing through an elongate subject, and converting the detected radiation into radiation image information, a first moving mechanism for moving the radiation source, a second moving mechanism for moving the radiation conversion panel, and a tomosynthesis control mechanism for moving the radiation source and the radiation conversion panel, which are disposed on the respective opposite sides of the subject, respectively in opposite directions in synchronism with each other; a image capturing range setting unit for setting a plurality of successive image capturing ranges in a longitudinal direction of the subject;_a longitudinal direction moving mechanism for moving an image capturing range of the tomosynthesis image capturing device in a longitudinal direction of the subject to allow the tomosynthesis image capturing device to capture the plurality of image capturing ranges such that the plurality of image capturing ranges are partly overlapped with each other;_an overlapping area specifying unit which acquires a plurality of radiation images of the plurality of image capturing ranges of an elongate test subject in advance, specifies overlapping areas in the radiation images, and registers information of the specified overlapping areas in an information table;_an image joining unit which acquires a plurality of radiation images of the plurality of image capturing ranges of the elongate subject, and joins the acquired plurality of radiation images based on the information of the overlapping areas registered in the information table to generate a single joined radiation image; and an image generating unit for generating a tomographic image on an arbitrary image plane based on the joined radiation image. The information of the overlapping areas may include a first relative number of pixels in a direction along the longitudinal direction and a second relative number of pixels in one direction perpendicular to the longitudinal direction or a direction opposite to the one direction.

The image joining unit may move any one of adjacent two radiation images in the longitudinal direction by the first relative number of pixels, move any one of the adjacent two radiation images in a direction perpendicular to the longitudinal direction by the second relative number of pixels, and then join the adjacent two radiation images.

The radiation image capturing apparatus may further include an input device for setting the image plane.

The tomosynthesis control mechanism of the tomosynthesis image capturing device may control the first moving mechanism and the second moving mechanism to move the radiation source and the radiation conversion panel in the longitudinal direction of the subject.

The tomosynthesis control mechanism of the tomosynthesis image capturing device may control the first moving mechanism and the second moving mechanism to move the radiation source and the radiation conversion panel in a direction perpendicular to the longitudinal direction of the subject.

The above and other objects, features, and advantages of the present invention will become more apparent from the following description when taken in conjunction with the accompanying drawings in which a preferred embodiment of the present invention is shown by way of illustrative example.

### BRIEF DESCRIPTION OF THE DRAWINGS

FIG. 1 is a block diagram of a radiation image capturing apparatus according to an embodiment of the present invention;

FIGS. 2A through 2C are side elevational views showing the manner in which a radiation source and a radiation conversion panel are moved in a longitudinal direction of a subject and a tomosynthesis image capturing apparatus is moved in the longitudinal direction of the subject;

FIGS. 3A through 3C are plan views showing the manner in which the radiation source and the radiation conversion panel are moved in the longitudinal direction of the subject and the tomosynthesis image capturing apparatus is moved in the longitudinal direction of the subject;

FIGS. 4A through 4C are plan views showing the manner in which the radiation source and the radiation conversion panel are moved in a direction perpendicular to the longitudinal direction of the subject and the tomosynthesis image capturing apparatus is moved in the longitudinal direction of the subject;

FIG. 5 is a block diagram of a circuit arrangement of the radiation conversion panel;

FIG. 6 is a view showing the manner in which three-dimensional images are joined together by a three-dimensional image joining unit; and

FIG. 7 is a view showing the manner in which the image plane of an elongate three-dimensional image is specified by an image generating unit.

### DESCRIPTION OF THE PREFERRED EMBODIMENT

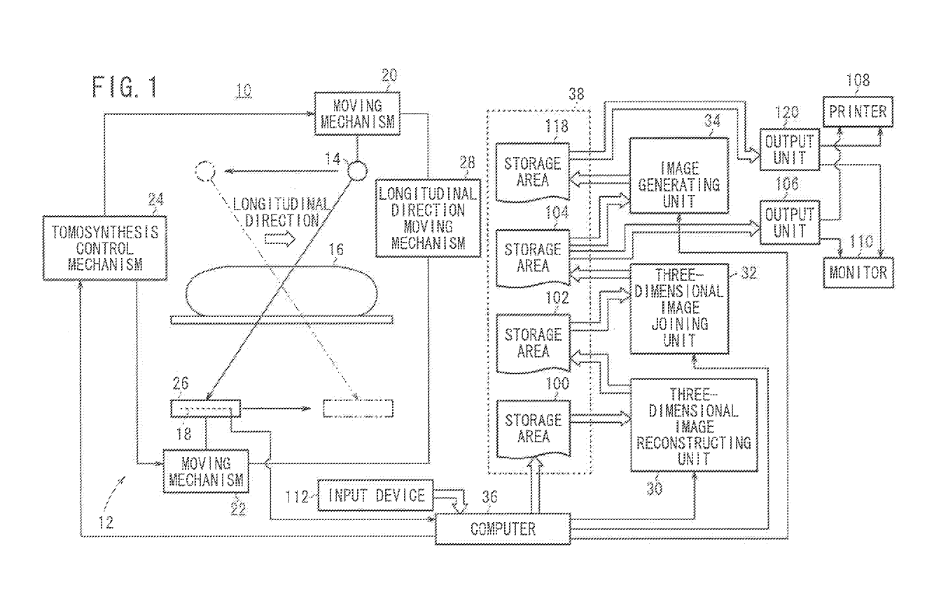

A radiation image capturing apparatus according to an embodiment of the present invention will be described below with reference to FIGS. 1 through 7.

As shown in FIG. 1, a radiation image capturing apparatus 10 according to an embodiment of the present invention includes a tomosynthesis image capturing device 12.

The tomosynthesis image capturing device 12 comprises a radiation source 14, a radiation conversion panel 18 for detecting a radiation emitted from the radiation source 14 and passing through an elongate subject 16, and converting the detected radiation into radiation image information, a first moving mechanism 20 for moving the radiation source 14, a second moving mechanism 22 for moving the radiation conversion panel 18, and a tomosynthesis control mechanism 24 for moving the radiation source 14 and the radiation conversion panel 18, which are disposed on the respective opposite sides of the subject 16, respectively in opposite directions in synchronism with each other. The radiation conversion panel 18 is housed in the casing of a cassette 26, for example. In the present embodiment, the tomosynthesis control mechanism 24 moves the radiation source 14 and the radiation conversion panel 18, which are disposed on the respective opposite sides of the subject 16, respectively in the opposite directions in synchronism with each other, such that a line interconnecting the center of the radiation source 14 and the center of the radiation conversion panel 18 is held in substantial alignment with the direction in which the radiation is emitted from the radiation source 14 to the subject 16.

The radiation image capturing apparatus 10 also includes, in addition to the tomosynthesis image capturing device 12, a longitudinal direction moving mechanism 28 for moving an image capturing range of the tomosynthesis image capturing device 12 in a longitudinal direction of the subject 16, a three-dimensional image reconstructing unit 30 for generating a single three-dimensional image from a plurality of radiation images captured in a single image capturing range, a three-dimensional image joining unit 32 for joining a plurality of three-dimensional images generated by the three-dimensional image reconstructing unit 30 with respect to a plurality of image capturing ranges, in the longitudinal direction of the subject 16, an image generating unit 34 for generating a two-dimensional projection image (tomographic image) on an arbitrary image plane based on a joined three-dimensional image produced by the three-dimensional image joining unit 32, and a computer 36 for controlling the above units 30, 32, 34.

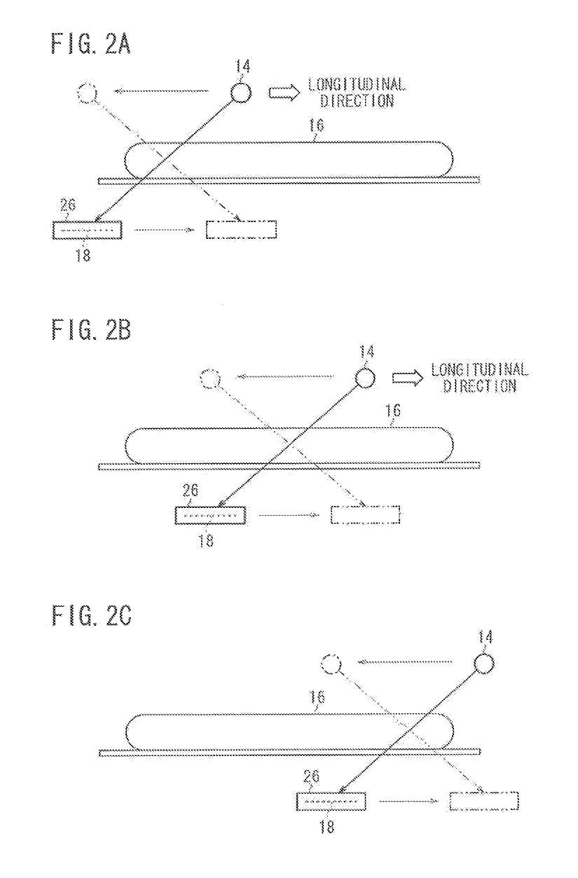

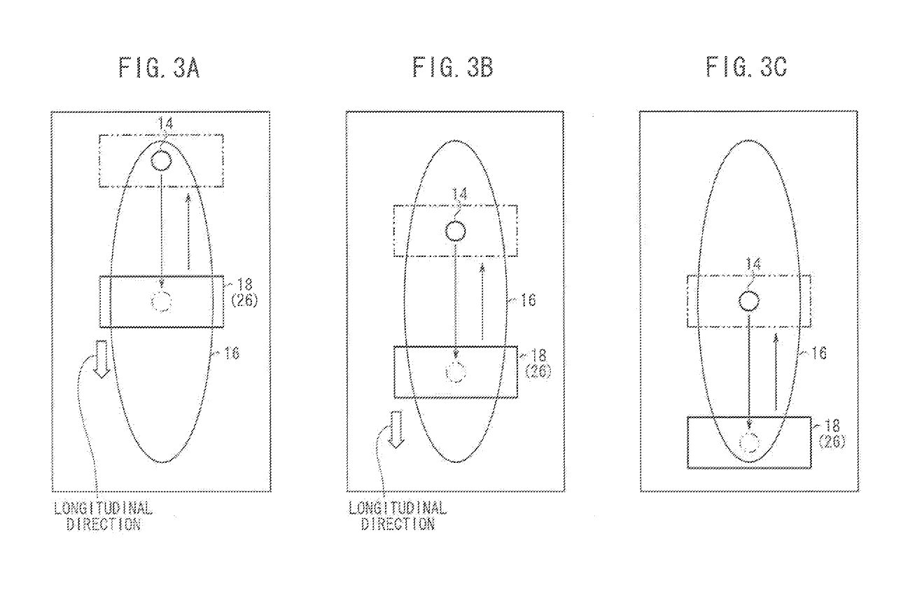

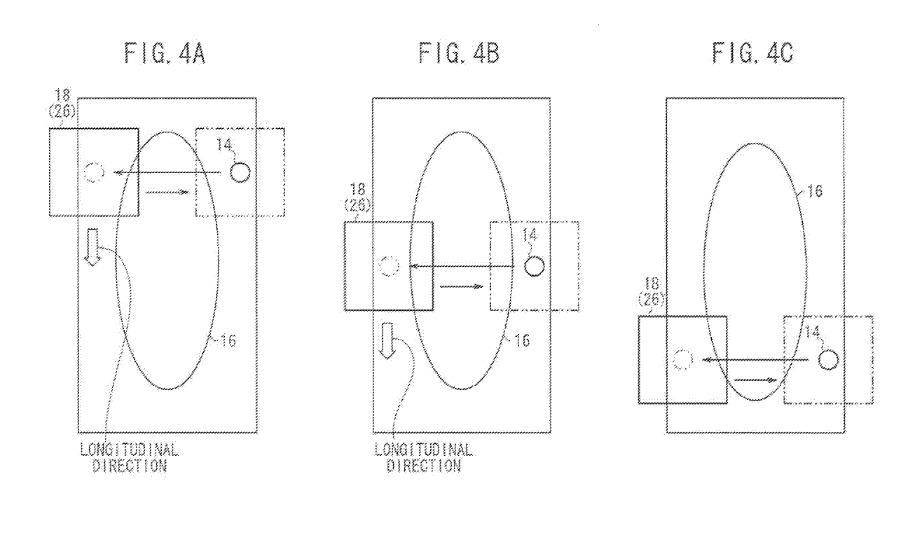

The tomosynthesis control mechanism 24 of the tomosynthesis image capturing device 12 controls the first moving mechanism 20 and the second moving mechanism 22 to move the radiation source 14 and the radiation conversion panel 18 (the cassette 26) in the longitudinal direction of the subject 16, as shown in FIGS. 2A through 2C and 3A through 3C. The tomosynthesis control mechanism 24 may also control the first moving mechanism 20 and the second moving mechanism 22 to move the radiation source 14 and the radiation conversion panel 18 in a direction perpendicular to the longitudinal direction of the subject 16, as shown in FIGS. 4A through 4C.

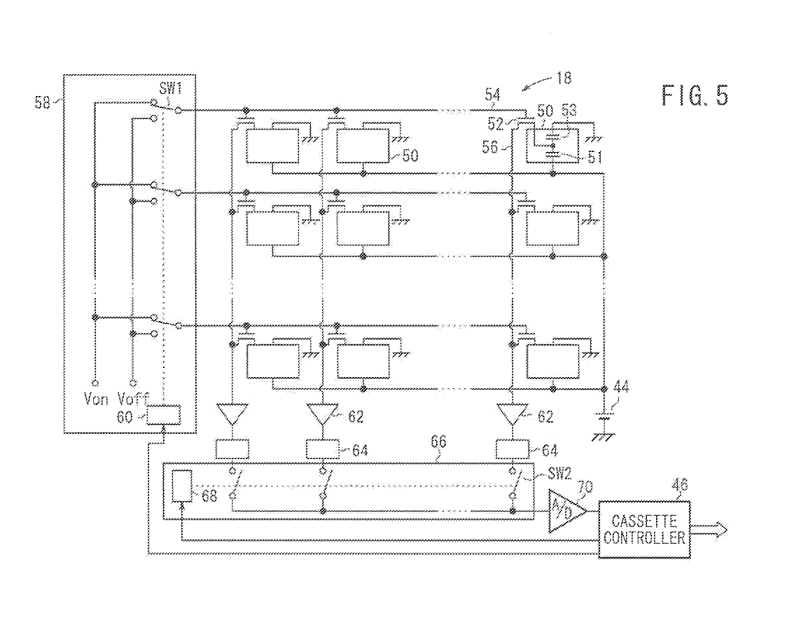

As shown in FIG. 5, the radiation conversion panel 18 comprises an array of thin-film transistors (TFTs) 52 arranged in rows and columns, a photoelectric conversion layer 51 made of a material such as amorphous selenium (a-Se) for generating electric charges upon detection of the radiation, the photoelectric conversion layer 51 being disposed on the array of TFTs 52, and an array of storage capacitors 53 connected to the photoelectric conversion layer 51. When the radiation X is applied to the radiation conversion panel 18, the photoelectric conversion layer 51 generates electric charges, and the storage capacitors 53 store the generated electric charges. Then, the TFTs 52 are turned on along each row at a time to read the electric charges from the storage capacitors 53 as an image signal. In FIG. 5, the photoelectric conversion layer 51 and one of the storage capacitors 53 are shown as a pixel 50, and the pixel 50 is connected to one of the TFTs 52. Details of the other pixels 50 are omitted from illustration. Since amorphous selenium tends to change its structure and lose its functionality at high temperatures, it needs to be used in a certain temperature range. Therefore, some means for cooling the radiation conversion panel 18 should preferably be provided in the cassette 26.

The TFTs 52 connected to the respective pixels 50 are connected to respective gate lines 54 extending parallel to the rows and respective signal lines 56 extending parallel to the columns. The gate lines 54 are connected to a line scanning driver 58, and the signal lines 56 are connected to a multiplexer 66 serving as a reading circuit.

The gate lines 54 are supplied with control signals Von, Voff for turning on and off the TFTs 52 along the rows from the line scanning driver 58. The line scanning driver 58 comprises a plurality of first switches SW1 for switching between the gate lines 54, and a column address decoder 60 for outputting a selection signal for selecting one of the first switches SW1 at a time. The column address decoder 60 is supplied with an address signal from the cassette controller 46.

The signal lines 56 are supplied with electric charges stored in the storage capacitors 53 of the pixels 50 through the TFTs 52 arranged in the columns. The electric charges supplied to the signal lines 56 are amplified by amplifiers 62 connected respectively to the signal lines 56. The amplifiers 62 are connected through respective sample and hold circuits 64 to the multiplexer 66. The multiplexer 66 comprises a plurality of second switches SW2 for switching between the signal lines 56, and a row address decoder 68 for outputting a selection signal for selecting one of the second switches SW2 at a time. The row address decoder 68 is supplied with an address signal from the cassette controller 46. The multiplexer 66 has an output terminal connected to an A/D converter 70. A radiation image signal generated by the multiplexer 66 based on the electric charges from the sample and hold circuits 64 is converted by the A/D converter 70 into a digital signal representing radiation image information, which is supplied to the cassette controller 46. The cassette controller 46 supplies the digital image signal to the computer 36, which stores the digital image signal, i.e., the radiation image information, in an image storage memory 38.

In other words, each time the tomosynthesis image capturing device 12 captures a radiation image of the subject 16, the radiation conversion panel 18 supplies radiation image information representing the radiation image to the computer 36, which stores the radiation image information in a first storage area 100 of the image storage memory 38.

Each time the tomosynthesis image capturing device 12 completes the capturing of images in an image capturing range, for example, the three-dimensional image reconstructing unit 30 reads out a plurality of pieces of radiation image information corresponding to the one image capturing range from the first storage area 100 of the image storage memory 38, reconstructs a single three-dimensional image from the read pieces of radiation image information according to a known three-dimensional image reconstructing algorithm, and stores the reconstructed three-dimensional image in a second storage area 102 of the image storage memory 38.

When the tomosynthesis image capturing device 12 completes the capturing of images in all image capturing ranges, therefore, the second storage area 102 of the image storage memory 38 stores reconstructed three-dimensional images corresponding to the respective image capturing ranges.

For imaging the elongate subject 16 in a plurality of image capturing ranges, it is preferable to set the image capturing ranges such that they partly overlap each other for the purpose of accurately imaging the elongate subject 16. Accordingly, the three-dimensional images generated in the adjacent two image capturing ranges include images of the overlapping areas. If the three-dimensional images are simply joined together according to the sequence of the image capturing ranges, then the images of the overlapping areas are juxtaposed in the joined part, and the resultant combined image is not accurately representative of the subject 16.

To avoid the above drawback, the three-dimensional image joining unit 32 specifies the overlapping areas. The overlapping areas may be specified by a first process based on a table of positional information, which has been recognized in advance, of the overlapping areas, or a second process based on pattern matching.

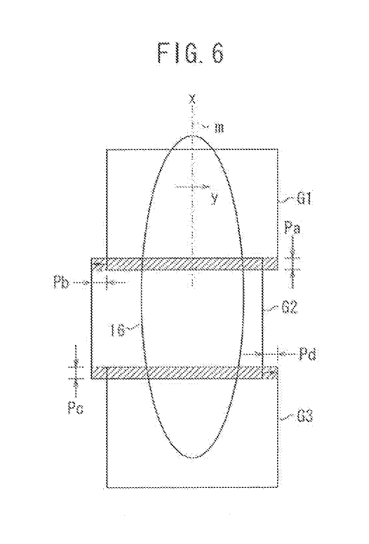

According to the first process, an elongate test subject is imaged to generate a plurality of three-dimensional images thereof, and the three-dimensional images are displayed on a display monitor. Then, the overlapping areas of the three-dimensional images are specified while the three-dimensional images are being moved on the display monitor using a CAD program or the like. After the overlapping areas are specified, information of the overlapping areas included in the three-dimensional images is registered in an information table. If the longitudinal direction of the subject 16 is indicated as an x direction (see FIG. 6) and the direction perpendicular to the longitudinal direction of the subject 16 is indicated as a y direction, then the information of the overlapping areas may include the relative number of pixels in the x direction, i.e., the relative number of overlapping pixels in the longitudinal direction of the subject 16, a sign (a positive sign or a negative sign) with respect to the y direction, and the relative number of pixels in the y direction. The positive sign represents a rightward direction from a central line m in the x direction, and the negative sign represents a leftward direction from the central line m in the x direction. FIG. 6 shows the manner in which a first three-dimensional image G1, a second three-dimensional image G2, and a third three-dimensional image G3 are joined together. In FIG. 6, the second three-dimensional image G2 is moved in the x direction by the relative number Pa of pixels and moved to the left by the relative number Pb of pixels, and is joined to the first three-dimensional image G1, and the third three-dimensional image G3 is moved in the x direction by the relative number Pc of pixels and moved to the right by the relative number Pd of pixels, and is joined to the second three-dimensional image G2. The overlapping areas of the three-dimensional images G1 through G3 are shown hatched in FIG. 6. The first process requires the test subject to be imaged and also requires the information table to be generated while the overlapping areas of the three-dimensional image are being confirmed on the monitor display. However, the first process is advantageous in that once the information table is generated, it can be used repeatedly until the next maintenance.

According to the second process, each of the pixels or each of a group of several pixels is regarded as a block, and pattern matching is carried out for each block. The pattern matching is readily applicable because it has been used to detect interframe motion vectors for image processing. Though the pattern matching is a simple process as there is no need for imaging a test subject and generating an information table while the overlapping areas of three-dimensional image are being confirmed on the monitor display, the pattern matching is problematic in that it requires time-consuming image processing.

The first process and the second process may be combined with each other. Specifically, an information table may be registered for rough ranges that may be regarded as overlapping areas according to the first process, and pattern matching is performed on the rough ranges according to the second process. The combination of the first and second processes makes it possible to specify the overlapping areas accurately at a high speed.

After the three-dimensional image joining unit 32 has specified the overlapping areas in each three-dimensional image, the three-dimensional image joining unit 32 calculates weighted averages of the pixels included in the overlapping areas. For example, the three-dimensional image joining unit 32 calculates weighted averages of the pixels included in a first overlapping area of a first three-dimensional image and a second three-dimensional image, for example, and generates a three-dimensional image of the first overlapping area based on the weighted averages. Then, the three-dimensional image joining unit 32 calculates weighted averages of the pixels included in a second overlapping area of the second three-dimensional image and a third three-dimensional image, for example, and generates a three-dimensional image of the second overlapping area based on the weighted averages. The three-dimensional image joining unit 32 calculates weighted averages of the pixels included in a third overlapping area of the third three-dimensional image and a fourth three-dimensional image, and generates a three-dimensional image of the third overlapping area based on the weighted averages. The three-dimensional image joining unit 32 operates similarly on other overlapping areas to generate three-dimensional images of the other overlapping areas.

Accordingly, the three-dimensional image joining unit 32 generates a single combined elongate three-dimensional image free of overlapping areas which is accurately representative of the elongate subject 16. The elongate three-dimensional image is stored in a third storage area 104 of the image storage memory 38. The elongate three-dimensional image stored in the third storage area 104 will be supplied to a printer 108 or a display monitor 110 via a first output unit 106.

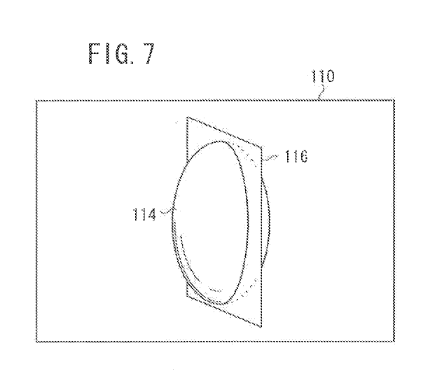

The image generating unit 34 generates a tomographic image on an arbitrary image plane based on the elongate three-dimensional image stored in the third storage area 104. The image plane is set using an input device 112 (a keyboard, a mouse, etc.) connected to the computer 36. If signals are entered via a GUI (Graphic User Interface), for example, then, as shown in FIG. 7, an image plane 116 is designated on an elongate three-dimensional image 114 displayed on the display monitor 110, as a sectional image across the elongate three-dimensional image 114, using a coordinate input device such as a mouse, for example. At this time, the image plane 116 may be specified by entering vertex coordinates. Alternatively, the image plane 116 may be specified simply by entering a distance from the radiation conversion panel 18.

When the image plane 116 is set, the image generating unit 34 determines a plurality of vertex coordinates where the elongate three-dimensional image 114 and the image plane 116 overlap each other based on coordinates and vectors on an image memory (VRAM) of the elongate three-dimensional image 114 and coordinates and vectors on an image memory (VRAM) of the image plane 116, and projects the elongate three-dimensional image 114 parallel onto the image plane 116 which is given as a plane specified by the vertex coordinates, thereby generating a two-dimensional image (tomographic image). The tomographic image is stored in a forth storage area 118 of the image storage memory 38. The tomographic image stored in the fourth storage area 118 will be supplied to the printer 108 or the display monitor 110 via a second output unit 120.

In addition to the above parallel projection, the image generating unit 34 is also capable of generating a two-dimensional image on an image plane according to a perspective transformation process having a viewpoint at the radiation source and a screen on the image plane, in response to a selective command entered via the input device. Specifically, based on preset world coordinates of the radiation source 14 and the world coordinates of the image plane 116 which are set as described above, the elongate three-dimensional image 114 is processed by a perspective transformation process having a viewpoint at the radiation source 14 and a screen on the image plane, thereby generating a tomographic image on the image plane 116. The tomographic image is different in overlapping and magnification from the elongate three-dimensional image 114 depending on the positional relationship between the radiation source 14, the subject 16, and the image plane 116. The tomographic image is also stored in the forth storage area 118 of the image storage memory 38. The tomographic image stored in the fourth storage area 118 will be supplied to the printer 108 or the display monitor 110 via the second output unit 120.

As described above, the radiation image capturing apparatus 10 according to the present embodiment comprises the longitudinal direction moving mechanism 28 for moving an image capturing range of the tomosynthesis image capturing device 12 in the longitudinal direction of the subject 16, the three-dimensional image reconstructing unit 30 for generating a single three-dimensional image from a plurality of radiation images captured in a single image capturing range, the three-dimensional image joining unit 32 for joining a plurality of three-dimensional images generated by the three-dimensional image reconstructing unit 30 with respect to a plurality of image capturing ranges, in the longitudinal direction of the subject 16, and the image generating unit 34 for generating a tomographic image on an arbitrary image plane 116 based on a joined three-dimensional image produced by the three-dimensional image joining unit 32. Using the three-dimensional image acquired in the tomosynthesis image capturing, the radiation image capturing apparatus 10 is capable of easily producing a tomographic image on the image plane 116 across the elongate subject 16, for thereby effectively shortening medical procedures that are performed using the radiation image capturing apparatus 10.

The radiation conversion panel 18 of the radiation image capturing apparatus 10 described above converts the incident radiation directly into an electrical signal corresponding to the amount of the radiation by means of the photoelectric conversion layer 51 (i.e., direct conversion type). Instead, the radiation conversion panel may, however, convert the incident radiation into visible light with a scintillator and then the visible light into an electrical signal using solid-state detecting elements such as a-Si (i.e., indirect conversion type, see Japanese Patent No. 3494683).

The radiation image information may also be acquired with light readout type radiation conversion panels. In light readout type radiation conversion panels, a two-dimensional array of solid-state detecting elements receives radiation and stores an electrostatic latent image corresponding to the amount of radiation. Readout of the latent image is carried out by applying reading light onto the radiation conversion panel and utilizing the value of the electrical current generated by the radiation conversion panel as radiation image information. After reading out the radiation image information, the latent image and the radiation image information of the radiation conversion panel can be erased by irradiating the radiation conversion panel with erasing light, so that the radiation conversion panel can be reused (see Japanese Laid-Open Patent Publication No. 2000-105297).

While the radiation conversion panel 18 of the radiation image capturing apparatus 10 utilizes the thin-film transistors (TFTs) 52 in the above-mentioned embodiments, such a device as a CCD (Charge Coupled Device), a CMOS (Complementary Metal Oxide Semiconductor) device or the like may also be used instead of the TFTs 52.

Although a certain preferred embodiment of the present invention has been shown and described in detail, it should be understood that various changes and modifications may be made therein without departing from the scope of the appended claims.

What is claimed is:

1. A radiation image capturing apparatus comprising:
a tomosynthesis image capturing device comprising a radiation source, a radiation conversion panel for detecting a radiation emitted from the radiation source and passing through an elongate subject, and converting the detected radiation into radiation image information, a first moving mechanism for moving the radiation source, a second moving mechanism for moving the radiation conversion panel, and a tomosynthesis control mechanism for moving the radiation source and the radiation conversion panel, which are disposed on the respective opposite sides of the elongate subject, respectively in opposite directions in synchronism with each other;
an image capturing range setting unit for setting a plurality of successive image capturing ranges in a longitudinal direction of the elongate subject;
a longitudinal direction moving mechanism for moving an image capturing range of the tomosynthesis image capturing device in the longitudinal direction of the elongate subject to allow the tomosynthesis image capturing device to capture the plurality of successive image capturing ranges, the plurality of successive image capturing ranges being partly overlapped with each other;
an overlapping area specifying unit which acquires a plurality of radiation images of an elongate test subject in advance in a plurality of successive image capturing ranges, specifies overlapping areas in the radiation images, and registers information of the specified overlapping areas in an information table;
an image joining unit which acquires a plurality of radiation images of the elongate subject in the plurality of successive image capturing ranges, and joins the acquired plurality of radiation images based on the information of the overlapping areas registered in the information table to generate a single joined radiation image; and
an image generating unit for generating a tomographic image on an arbitrary image plane based on the joined radiation image.

2. The radiation image capturing apparatus according to claim 1, the information of the overlapping areas comprise a first relative number of pixels in a direction along the longitudinal direction and a second relative number of pixels in one direction perpendicular to the longitudinal direction or a direction opposite to the one direction.

3. The radiation image capturing apparatus according to claim 2, the image joining unit moves any one of adjacent two radiation images in the longitudinal direction by the first relative number of pixels, moves any one of the adjacent two radiation images in a direction perpendicular to the longitudinal direction by the second relative number of pixels, and then joins the adjacent two radiation images.

4. The radiation image capturing apparatus according to claim 1, further comprising an input device for setting the image plane.

5. The radiation image capturing apparatus according to claim 1, wherein the tomosynthesis control mechanism of the tomosynthesis image capturing device controls the first moving mechanism and the second moving mechanism to move the radiation source and the radiation conversion panel in the longitudinal direction of the subject.

6. The radiation image capturing apparatus according to claim 1, wherein the tomosynthesis control mechanism of the tomosynthesis image capturing device controls the first moving mechanism and the second moving mechanism to move the radiation source and the radiation conversion panel in a direction perpendicular to the longitudinal direction of the subject.
